(19) United States
(12) Patent Application Publication
(10) Pub. No.: US 2009/0215806 A1
(43) Pub. Date: Aug. 27, 2009

(54) Rosuvastatin calcium with a low salt content

(76) Inventors: Valerie Niddam-Hildesheim, Kadima (IL); Shalom Shabat, Yavne (IL); Dalia Maidan-Hanoch, Kfar Yona (IL)

Correspondence Address: KENYON & KENYON LLP, ONE BROADWAY, NEW YORK, NY, 10004, US

(21) Appl. No.: 12/387,634

(22) Filed: May 4, 2009

Related U.S. Application Data
(62) Division of application No. 11/506,062, filed on Aug. 16, 2006.
(60) Provisional application No. 60/709,065, filed on Aug. 16, 2005.

(51) Int. Cl. A61K 31/505; C07D 239/42

(52) U.S. Cl. 514/275; 544/297

(57) ABSTRACT

Provided is rosuvastatin calcium with a low salt by product content and processes for preparing such rosuvastatin calcium.

### RELATED APPLICATIONS

This application claims the benefit of provisional application Ser. No. 60/709,065, filed Aug. 16, 2005, which is incorporated herein by reference.

### FIELD OF INVENTION

The invention relates to rosuvastatin calcium with a low salt content and a process for preparing such rosuvastatin calcium.

### BACKGROUND OF THE INVENTION

Statins are currently the most therapeutically effective drugs available for reducing low-density lipoprotein (LDL) particle concentration in the blood stream of patients at risk for cardiovascular disease. Thus, statins are used in the treatment of hypercholesterolemia, hyperlipoproteinemia, and atherosclerosis. A high level of LDL in the bloodstream has been linked to the formation of coronary lesions that obstruct the flow of blood and can rupture and promote thrombosis. Goodman and Gilman, The Pharmacological Basis of Therapeutics, p. 879 (9th Ed. 1996).

Rosuvastatin calcium (monocalcium bis (+) 7-[4-(4-fluorophenyl)-6-isopropyl-2-(N-methyl-N-methylsulfonylaminopyrimidin)-5-yl]-(3R,5S)-dihydroxy-(E)-6-heptenoate) is an HMG-CoA reductase inhibitor, developed by Shionogi for the once daily oral treatment of hyperlipidaemia (Ann Rep, Shionogi, 1996; Direct communications, Shionogi, 8 Feb. 1999 & 25 Feb. 2000). Rosuvastatin calcium is a superstatin, which can lower LDL-cholesterol and triglycerides more effectively than first generation drugs. Rosuvastatin calcium has the following chemical formula:

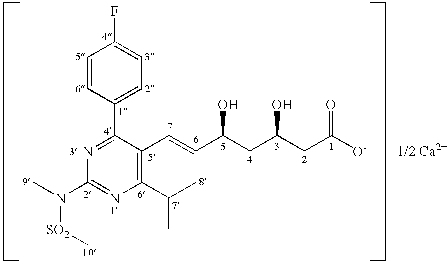

Rosuvastatin calcium is marketed under the name CRESTOR for treatment of a mammal such as a human. According to the maker of CRESTOR, it is administered in a daily dose of from about 5 mg to about 40 mg to lower LDL cholesterol levels.

U.S. Pat. No. 5,260,440 discloses the preparation of rosuvastatin by reacting 4-(4-fluorophenyl)-6-isopropyl-2-(N-methyl-N-methylsulfonylamino)-5-pyrimidinecarbaldehyde with methyl (3R)-3-(tert-butyldimethylsilyloxy)-5-oxo-6-triphenylphosphoranylidene hexanate in acetonitrile under reflux. The silyl group is then cleaved with hydrogen fluoride, followed by reduction with NaBH4 and diethylmethoxyborane in THF to obtain a methyl ester of rosuvastatin.

The ester is then hydrolyzed with sodium hydroxide in ethanol at room temperature, followed by removal of ethanol and addition of ether to obtain the sodium salt of rosuvastatin. The sodium salt is then converted to the calcium salt by dissolving the sodium salt in water under a nitrogen atmosphere. Calcium chloride is then added to the solution, resulting in precipitation of rosuvastatin calcium (2:1).

The method of the '440 patent, as well as WO 04/108691, produce the calcium salt of rosuvastatin through the formation of an intermediate salt, such as a sodium salt, using an aqueous alkali metal hydroxide. The sodium salt is then converted to a calcium salt by using calcium chloride. This chemical reaction may produce high concentrations of sodium chloride (at least one equivalent). The following schemes illustrate the reactions:

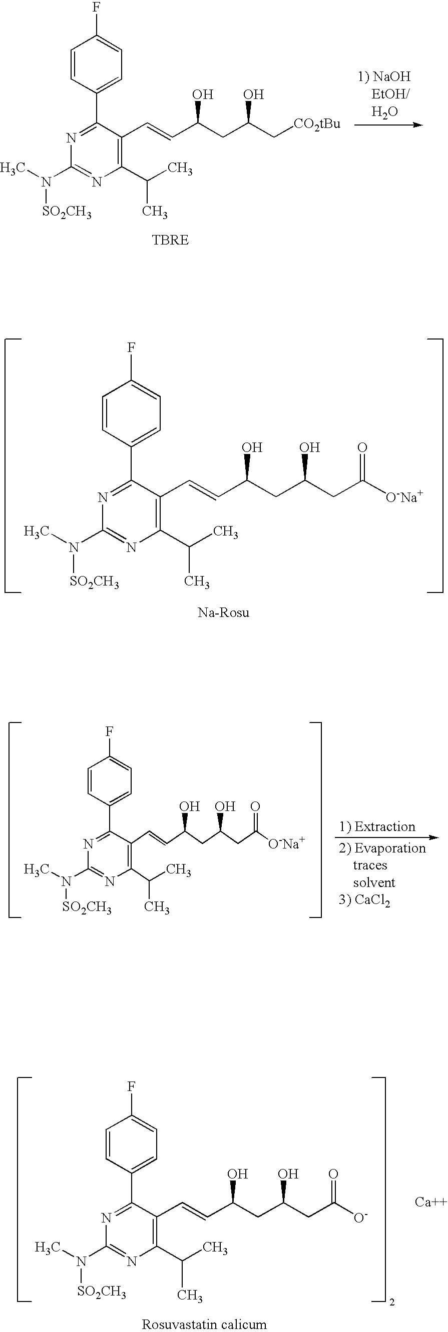

The Applicants have found that the rosuvastatin calcium salt precipitates in aggregates. In particular, the Applicants have found that the aggregates enclose the resulting salt by product and prevent it from being removed by regular techniques.

### SUMMARY OF THE INVENTION

In one embodiment, the invention provides a process of reducing levels of salt by-products present in a composition of rosuvastatin calcium and the salt by products comprising the steps of: a) providing a composition having rosuvastatin calcium and salt by products; and b) physically breaking up the composition in presence of water to reduce the level of the salt by product in the composition.

In another embodiment, the invention provides a process of reducing formation of rosuvastatin calcium aggregates containing salt by products comprising the steps of:

a) combining a catalytic amount of sodium borohydride with an aqueous reaction mixture containing a C₁ to C₄ alkyl ester of rosuvastatin;

b) adding a base to the reaction mixture to hydrolyze the ester;

c) adding a source of calcium to the hydrolyzed ester to precipitate rosuvastatin calcium.

Another embodiment of the invention provides a process of reducing levels of salt by-products present in a composition of rosuvastatin calcium and the salt by products comprising the steps of:

a) providing a reaction mixture of a C₁ to C₄ alkyl ester of rosuvastatin;

b) hydrolyzing thy ester with sodium or potassium hydroxide, thereby forming a rosuvastatin sodium or potassium salt;

c) adding calcium chloride or calcium acetate to obtain a composition of rosuvastatin calcium and salt by products; and

d) breaking up the composition in presence of water to reduce levels of the salt by product in the composition.

One embodiment of the invention provides a process of reducing formation of rosuvastatin calcium aggregates comprising the steps of:

a) providing a reaction mixture of a C₁ to C₄ alkyl ester of rosuvastatin;

b) adding a catalytic amount of sodium borohydride to the reaction mixture;

c) hydrolyzing the ester with sodium or potassium hydroxide, thereby forming a sodium or potassium salt; and

d) adding calcium chloride or calcium acetate to the sodium or potassium salt, thereby precipitating rosuvastatin calcium.

In a further embodiment, the invention provides rosuvastatin calcium produced by the processes of the invention.

Another embodiment of the invention provides rosuvastatin calcium having a salt by product content of less than about 0.1% by weight.

A further embodiment of the invention provides rosuvastatin calcium having a chloride content of less than about 0.1% by weight.

One embodiment of the invention provides rosuvastatin calcium having an acetate content of less than about 0.1% by weight.

Another embodiment of the invention provides a pharmaceutical composition containing an effective amount of rosuvastatin calcium substantially free of salt by products in combination with a pharmaceutically acceptable excipient.

Also provided is a process of preparing a pharmaceutical composition containing an effective amount of rosuvastatin calcium substantially free of salt by products including the step of combining rosuvastatin calcium having a salt by product content of less than about 0.1% by weight with a pharmaceutically acceptable excipient.

One embodiment of the invention provides a method of treating a mammal in need of inhibition of the 3-hydroxy-3-methyl-glutaryl-coenzyme A (“HMG-CoA”) reductase enzyme including administering a pharmaceutical composition containing an effective amount of rosuvastatin calcium substantially free of salt by products to the mammal.

### DETAILED DESCRIPTION OF THE INVENTION

The invention provides rosuvastatin calcium substantially free of salt by products. One embodiment provides for a calcium salt of rosuvastatin with a salt by product level of less than about 0.1%, preferably less than about 0.05% and more preferably less than about 0.03% by weight (weight measures provided herein are based on the anionic component of the salt).

The salt is a by-product of the ion exchange reaction in the last step of making rosuvastatin calcium, in which the sodium in rosuvastatin sodium forms a salt with the anion of the calcium source. Salt by products of the ion exchange reaction can include a salt formed by reaction of a cation, such as an alkali metal or alkaline earth metal (other than calcium), with an anion from the calcium salt used in the ion exchange step. Preferably, the by product salt is sodium chloride or sodium acetate. More preferably, the salt by product is sodium chloride.

A precipitate is obtained upon forming rosuvastatin calcium in a reaction mixture. The precipitate is a composition (i.e., mixture) of rosuvastatin calcium and salt by products since rosuvastatin calcium precipitates as aggregates. These aggregates of rosuvastatin calcium sequester the salt by product within them. As a result, the salts cannot dissolve in the reaction mixture and remain in the composition.

When synthesizing rosuvastatin calcium from an ester of rosuvastatin, the first step involves the hydrolysis of a C₁-C₄ alkyl ester of rosuvastatin to obtain a salt (for example, the sodium salt is obtained when sodium hydroxide is used in hydrolysis) (see, e.g., WO2005023778). Hydrolysis is preferably carried out in an aqueous solvent in the presence of a base. The hydrolysis results in an aqueous solution of a salt of rosuvastatin, such as the sodium salt. The resulting solution can then be optionally washed with a water immiscible solvent, such as toluene, to extract impurities including reagents. Active carbon can be used to further purify the solution. A water soluble calcium salt is added to the rosuvastatin sodium salt solution causing an exchange of calcium and sodium, and resulting in precipitation of rosuvastatin calcium. Rosuvastatin calcium salt can be recovered, for example, by filtration. Rosuvastatin acetate may be prepared in a similar fashion with the use of a source of acetate ions, such as sodium acetate.

To obtain rosuvastatin calcium with a by product salt content of less than about 0.1% by weight, a number of different approaches were taken. These approaches included:
varying the feeding time of calcium salt during synthesis;
varying the source of calcium, such as by adding calcium acetate as the calcium salt;
adding the calcium salt as a liquid or a solid;
varying the pH of the solution before extraction;
varying the number of washings of the precipitate;
wet milling the precipitate;
a catalytic amount of sodium borohydride (NaBH₄); and
compressing a wet cake of rosuvastatin calcium.

Of these approaches, the last three resulted in a reduced concentration of salt by product in the final material, with wet milling and use of a catalytic amount of sodium borohydride being particularly effective in reducing the concentration of the salt by-product present in a composition of rosuvastatin calcium and the salt by product.

The addition time of calcium salt does not produce a significant difference in salt by product concentration of the final material. As illustrated in Table 1, there is no difference in salt by product concentration when calcium chloride is added over five minutes or when calcium chloride is added over two hours.

TABLE 1
Influence of the addition time of CaCl₂ on the
salt by product content of the final material

| | Level of |
|---|---|
| Description | chlorides (%) |
| Addition of CaCl₂ | 0.10 |
| during 5 minutes | |
| Addition of CaCl₂ | 0.10 |
| during 1-2 hours | |

The calcium salt may be added as a solid or a liquid. As illustrated in Table 2, the state in which the calcium salt is added does not produce a significant reduction in the salt by product concentration of the final material.

TABLE 2
Influence of CaCl₂ form on the level of
salt by product in the final material

| | Level of |
|---|---|
| Description | chlorides (%) |
| Addition of CaCl₂ | 0.10 |
| Solid | |
| Addition of CaCl₂ | 0.13 |
| 2N | |

As illustrated in Table 3, extraction of the solution at different pH values does not influence the salt by product concentration of the final material.

TABLE 3
Influence of the pH on the level of salt
by product in the final material

| Level of pH before | Level of |
|---|---|
| extraction | chlorides (%) |
| 12.6 | 0.10 |
| 10 | 0.17 |
| 8.5 | 0.13 |

Sodium salts are highly soluble in water and thus the salt by product content may be reduced to some degree by washings with water. After the first washing, however, there are diminishing returns from increasing the number of washings. Although the washing with water does allow the free salt by product to be removed, the salt by product in the aggregate structure is protected from being removed in this manner. Such diminishing return is illustrated in Table 4. An alternative method is necessary to release the salt by product from the aggregate to obtain a reduced salt by product concentration in the final product.

TABLE 4
Influence of washing with water on the salt
by product level in the final material

| Number of | Level of |
|---|---|
| washings | chlorides (%) |
| without | 0.52 |
| 1 | 0.29 |
| 2 | 0.21 |
| 3 | 0.19 |
| 4 | 0.18 |
| 5 | 0.15 |
| 6 | 0.14 |

As illustrated in table 5, using a different calcium source, such as calcium acetate rather than calcium chloride, did not address the problem.

TABLE 5
Influence of using a different calcium source

| | Level of acetic | Level of |
|---|---|---|
| Description | acid | sodium |
| Use of Ca(OAc)₂ | 0.44% | 0.17% |

The level of acetic acid was determined by titration, and the level of sodium by ICP (Inductively Coupled Plasma) analysis of the final material. The levels detected corresponded to a high level of sodium acetate salt contamination.

Breaking up aggregates by applying a force to wet rosuvastatin calcium, i.e., rosuvastatin calcium in the presence of water, effectively reduces the salt by product content of the final product. Preferably, the force is applied by milling or compressing wet rosuvastatin calcium. The process can be carried out with a slurry (heterogeneous mixture), such as a wet cake of rosuvastatin calcium obtained from precipitation.

The wet milling process allows the aggregate formed by the rosuvastatin calcium salt to be broken up so that water can interact with the salt by product. In wet milling, the solid is ground in the presence of water. On a laboratory scale, wet milling can be carried out using a Ultra Turrax T-25 from IKA with a rosuvastatin calcium present as a heterogeneous mixture in water (slurry). Since salt by products such as sodium chloride are highly soluble in water, wet milling can remove a significantly higher amount of salt by products than water washing alone. This results in a rosuvastatin calcium salt with a reduced concentration of salt by product, such as chloride, in the final material.

The wet milling process is preferably carried out at a temperature of about 0° C. to about 50° C., and more preferably at about 20° C. to about 30° C. The milling, depending on the scale involved in the process, preferably lasts about 1 minute to about 2 hours, more preferably about 10 minutes. Table 6, below, illustrates the influence of wet milling on the salt by product content of a composition of rosuvastatin calcium and the salt product.

The level of salts is also reduced by compressing wet rosuvastatin calcium, for example, by centrifuging a wet cake of a composition of rosuvastatin calcium and salt by product at high speeds, such as above about 100 rpm, including about 1000 rpm.

TABLE 6
Influence of wet milling on the chloride
content in the final material

| | A level of |
|---|---|
| Description | chlorides |
| Wet Milling | 0.01 |

The level of by product salts is also reduced by using a catalytic amount of a hydride. The proper systematic name for the compound NaBH₄ is sodium tetrahydridoborate. However, it is also called by the shorter name sodium borohydride. In the process of the invention, a catalytic amount of sodium borohydride is combined with an aqueous reaction mixture containing a C₁ to C₄ alkyl ester of rosuvastatin, followed by addition of a base to the mixture to start the hydrolysis process. A source of calcium is then added to precipitate rosuvastatin calcium.

A catalytic amount of a hydride is used in the process of the invention. A “catalytic amount” generally refers to an amount of less than about 10 mole percent. The catalytic amount is preferably of about 0.01% to about 5% by weight relative to the alkyl ester of rosuvastatin, more preferably about 0.05% by weight. The sodium borohydride influences the precipitation of rosuvastatin calcium to prevent aggregates of rosuvastatin calcium salt being formed. Without aggregates of rosuvastatin calcium, the salt by product, such as sodium chloride, can interact freely with the water and be removed. Table 7 illustrates the effectiveness of using sodium borohydride in the method of the invention.

TABLE 7
Effect of NaBH₄ on the chloride content in the final material

| | level of |
|---|---|
| Description | chlorides |
| Use NaBH₄ | 0.02 |

As one of skill in the art would appreciate, the methods of the invention, such as use of wet milling and a catalytic amount of sodium borohydride, can be combined to further reduce the level of impurities. In another embodiment, the combination of sodium borohydride, milling and centrifuging is used. In yet another embodiment, the combination of sodium borohydride and centrifuging is used.

Another embodiment of the invention encompasses pharmaceutical compositions comprising rosuvastatin calcium substantially free of salt by products, and at least one pharmaceutically acceptable excipient.

A further embodiment encompasses a process for preparing a pharmaceutical formulation comprising combining rosuvastatin calcium substantially free of salt by products, with at least one pharmaceutically acceptable excipient.

Another embodiment of the invention encompasses the use of rosuvastatin calcium substantially free of salt by products in the manufacture of a pharmaceutical composition.

Pharmaceutical formulations/compositions of the invention contain rosuvastatin calcium substantially free of salt by products. The rosuvastatin calcium prepared by the processes of the invention is ideal for pharmaceutical formulation. In addition to the active ingredient(s), the pharmaceutical formulations of the invention may contain one or more excipients. Excipients are added to the formulation for a variety of purposes.

Diluents increase the bulk of a solid pharmaceutical formulation, and may make a pharmaceutical dosage form containing the formulation easier for the patient and care giver to handle. Diluents for solid formulations include, for example, microcrystalline cellulose (e.g. Avicel® and beta form), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical formulations that are compacted into a dosage form, such as a tablet, may include excipients whose functions include helping to bind the active ingredient and other excipients together after compression. Binders for solid pharmaceutical formulations include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical formulation in the patient's stomach may be increased by the addition of a disintegrant to the formulation. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid formulation and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered formulation, the formulation is subjected to pressure from a punch and dye. Some excipients and active ingredients have a tendency to adhere to the surfaces of the punch and dye, which can cause the product to have pitting and other surface irregularities. A lubricant can be added to the formulation to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the formulation of the present invention include maltol, vanillin, ethyl vanillin, menthol, citric acid, fumaric acid, ethyl maltol and tartaric acid.

Solid and liquid formulations may also be dyed using any pharmaceutically acceptable colorant to improve their appearance and/or facilitate patient identification of the product and unit dosage level In liquid pharmaceutical formulations of the present invention, valsartan and any other solid excipients are dissolved or suspended in a liquid carrier such as water, vegetable oil, alcohol, polyethylene glycol, propylene glycol or glycerin.

Liquid pharmaceutical formulations may contain emulsifying agents to disperse uniformly throughout the formulation an active ingredient or other excipient that is not soluble in the liquid carrier. Emulsifying agents that may be useful in liquid formulations of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical formulations of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the invention, a liquid formulation may also contain a buffer such as guconic acid, lactic acid, citric acid or acetic acid, sodium guconate, sodium lactate, sodium citrate or sodium acetate. Selection of excipients and the amounts used may be readily determined by the formulation scientist based upon experience and consideration of standard procedures and reference works in the field.

Solid formulations of the invention include powders, granulates, aggregates and compacted formulations. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs.

The dosage form of the invention may be a capsule containing the formulation, preferably a powdered or granulated solid formulation of the invention, within either a hard or soft shell. The shell may be made from gelatin and optionally contain a plasticizer such as glycerin and sorbitol, and an opacifying agent or colorant.

The active ingredient and excipients may be formulated into formulations and dosage forms according to methods known in the art.

A formulation for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired-particle size. The granulate may then be tableted, or other excipients may be added prior to tableting, such as a glidant and/or a lubricant.

A tableting formulation may be prepared conventionally by dry blending. For example, the blended formulation of the actives and excipients may be compacted into a slug or a sheet and then comminuted into compacted granules. The compacted granules may subsequently be compressed into a tablet.

As an alternative to dry granulation, a blended formulation may be compressed directly into a compacted dosage form using direct compression techniques. Direct compression produces a more uniform tablet without granules. Excipients that are particularly well suited for direct compression tableting include microcrystalline cellulose, spray dried lactose, di-calcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

In addition to excipients, the pharmaceutical formulations of the present invention may contain an adjuvant.

Having described the invention with reference to certain preferred embodiments, other embodiments will become apparent to one skilled in the art from consideration of the specification. The invention is further defined by reference to the following examples describing in detail the preparation of the compound of the present invention. It will be apparent to those skilled in the art that many modifications, both to materials and methods, may be practiced without departing from the scope of the invention.

### Chloride Content in Rosuvastatin Calcium (By Titration)

Weigh accurately about 1.0 g of Rosuvastatin Ca, dissolve in 20 ml of DMSO and sonicate until fully dissolved.

Add about 40 ml H₂O, stir well and add 1 ml Of 10% HNO₃ to obtain a transparent solution.

Titrate potenciometrically with 0.01N AgNO₃.

### Acetate Content in Rosuvastatin Calcium (By HPLC)

HPLC condition:

Column—C8

Mobile phase—Gradient of Eluent A and Eluent B

Eluent A—95% (0.1% v/v H3PO4 in H2O): 5% MeOH

Eluent B—1:1 MeOH:ACN

Gradient

| Time | Eluent A | Eluent B |
|---|---|---|
| 0 | 100 | 0 |
| 5 | 100 | 0 |
| 8 | 10 | 90 |
| 16 | 10 | 90 |

### EXAMPLE 1

### Feeding of CaCl₂ During 1-2 Hours

A 1000 ml reactor equipped with a mechanical stirrer was charged with EtOH (200 mL), water (120 ml), and t-Butyl-Rosuvastatin (40 g), forming a suspension. To this suspension, NaOH 47% 1.2eq (7.6 g) was added dropwise at 25±5° C. Water (280 ml) was added, forming a mixture. The mixture was then washed with Toluene (200 ml) and stirred at 25±5° C. for half an hour, creating an aqueous layer.

The aqueous layer was isolated, active carbon was added, stirred at 25±5° C. for 30 min. and then filtered under reduced pressure with Sinter. Thereafter this solution was concentrated under reduced pressure at 40° C. to half the solution volume. Make-up of the solution was performed to 10 volumes of water versus TBRE (t-butyl rosuvastatin ester). The solution was then heated to 40-45° C. CaCl₂ (4.1 g) was added portionwise to this solution over 1-2 hour at 38-45° C., forming a suspension. The suspension was then cooled to 25±5° C., stirred at 25±5° C. for 1 hr, filtered and washed with water (3×20 ml) to get a powdery compound (17 g dry, 92%, Chloride content 0.1% by weight).

### EXAMPLE 2

### Feeding of CaCl₂ During 5 Min

A 1000 ml reactor equipped with a mechanical stirrer was charged with EtOH (200 mL), water (120 ml), and t-Butyl-Rosuvastatin (40 g), forming a suspension. To this suspension, NaOH 47% 1.2 eq (7.6 g) was added dropwise at 25±5° C., forming a mixture. The mixture was stirred at 25±5° C. for two hours. Active carbon was added and the mixture was stirred at 25±5° C. for 30 min. Water (280 ml) was then added. The mixture was then washed with Toluene (200 ml) and stirred at 25+5° C. for half an hour.

The aqueous layer was isolated. Thereafter the solution (i.e., the aqueous layer) was concentrated under reduced pressure-at 40° C. to half the solution volume. Make-up of the solution was performed to 10 volumes of water versus TBRE. The solution was heated to 40-45° C. CaCl₂ (4.1 g) was added portionwise to this solution over 5 min at 38-45° C., forming a suspension. The suspension was then cooled to 25±5° C., stirred at 25±5° C. for 1 hr, filtered and washed with water (3×20 ml) to get a powdery compound (16.4 g dry, 88%, Chloride content 0.1% by weight).

### EXAMPLE 3

Feeding with CaCl₂ Solid

A 1000 ml reactor equipped with a mechanical stirrer was charged with EtOH (200 mL), water (120 ml), and t-Butyl-Rosuvastatin (40 g), forming a suspension. To this suspension, NaOH 47% 1.2 eq (7.6 g) was added dropwise at 25±5° C. Water (280 ml) was added, forming a mixture. The mixture was then washed with Toluene (200 ml) and stirred at 25±5° C for half an hour. The aqueous layer was isolated.

To this solution (i.e., the aqueous layer) active carbon was added, the solution was stirred at 25±5° C. for 30 min. and then filtered under reduced pressure with Sinter. Thereafter the solution was concentrated under reduced pressure at 40° C. to half the solution volume. Make-up of the solution was performed to 10 volumes of water versus TBRE. The solution was heated to 40-45° C. CaCl₂ (4.1 g) was added to this solution over 1-2 hours at 38-45° C., forming a suspension. The suspension was then cooled to 25±5° C., stirred at 25±5° C. for 1hr, filtered and washed with water (3×20 ml) to get a powdery compound (17 g dry, 92%, Chloride content 0.1% by weight).

### EXAMPLE 4

### Feeding With a Solution of CaCl₂ 2N

A 1000 ml reactor equipped with a mechanical stirrer was charged with EtOH (200 mL), water (120 ml), and t-Butyl-Rosuvastatin (40 g), forming a suspension. To this suspension, NaOH 47% 1.2 eq (7.6 g) was added dropwise at 25±5° C., forming a mixture. The mixture was stirred at 25±5° C. for two hours. To this mixture active carbon was added and the mixture was stirred at 25±5° C. for 30 min. Water (280 ml) was added, and the mixture was then washed with Toluene (200 ml) and stirred at 25±5° C. for half an hour.

The aqueous layer was isolated and filtered under reduced pressure with Synter and Hyflo. Thereafter the solution was concentrated under reduced pressure at 40° C. to half the solution volume. Make-up of the solution was performed to 10 volumes of water versus TBRE. The solution was then heated to 40-45° C. CaCl₂ 2N (4.1 g+20 ml water) was added dropwise to the aqueous phase over 1 hour at 38-45° C., forming a suspension. The suspension was then cooled to 25±5° C., stirred at 25±5° C. for 1 hr, filtered and washed with water (3×20 ml) to get a powdery compound (18.1 g dry, 95%, Chloride content 0.1% by weight).

### EXAMPLE 5

### Level of pH Before Extraction-12.6

A 1000 ml reactor equipped with a mechanical stirrer was charged with EtOH (200 mL), water (120 ml), and t-Butyl-Rosuvastatin (40 g), forming a suspension. To this suspension, NaOH 47% 1.2 eq (7.6 g) was added dropwise at 25±5° C. Water (280 ml) was added, forming a mixture. The mixture was then washed with Toluene (200 ml) and stirred at 25±5° C. for half an hour.

The aqueous layer was isolated. To this solution active carbon was added and the mixture was stirred at 25±5° C. for 30 min. and then filtered under reduced pressure with Sinter. Thereafter the solution was concentrated under reduced pressure at 40° C. to half the solution volume. Make-up of the solution was performed to 10 volumes of water versus TBRE. The solution was heated to 40-45° C. CaCl₂ (4.1 g) was added dropwise to this solution over 1-2 hour at 38-45° C., forming a suspension. The suspension was then cooled to 25±5° C., stirred at 25±5° C. for 1 hr, filtered and washed with water (3±20 ml) to get a powdery compound (17 g dry, 92%, Chloride content 0.1% by weight).

### EXAMPLE 6

### Level of pH Before Extraction-10

A 1000 ml reactor equipped with a mechanical stirrer was charged with EtOH (100 mL), water (60 ml), t-Butyl-Rosuvastatin (20 g), and Carbon active (2 g), forming a suspension.

To this suspension, NaOH 47% 1.1 eq (3.5 g) was added dropwise at 25±5° C., forming a mixture. The mixture was stirred at 25±5° C. for two hours. The mixture was filtered under reduced pressure with Sinter to eliminate the Carbon active present. To this mixture water (140 ml) was added and the mixture was then acidified with HCl 0.1M until pH 10. The mixture was washed with Toluene (100 ml) and stirred at 25±5° C. for half an hour.

The aqueous layer was isolated and concentrated under reduced pressure at 40° C. to half the solution volume. Make-up of the solution was performed to 10 volumes of water versus TBRE. The solution was then heated to 40-45° C. CaCl₂ (4.13 g) was added dropwise to this solution over 30-90 min at 38-45° C., forming a suspension. The suspension was then cooled to 25±5° C., stirred at 25±5° C. for 1 hr, filtered and washed with water (4×20 ml) to get a powdery compound (17.5 g dry, 93%, Chloride content 0.17% by weight).

### EXAMPLE 7

### Level of pH Before Extraction-8.5

A 1000 ml reactor equipped with a mechanical stirrer was charged with EtOH (9 2 mL), water (55 ml), t-Butyl-Rosuvastatin (18.4 g), and active carbon (2 g), forming a suspension. To this suspension, NaOH 47% 1.1 eq (1.48 g) was added dropwise at 25±5° C., forming a mixture. The mixture was stirred at 25±5° C. for two hours. To this mixture water (129 ml) was added and the mixture was acidified with HCl 0.1M until pH 8.5. The mixture was then washed with Toluene (92 ml) and stirred at 25±5° C. for half an hour.

The aqueous layer was isolated and was concentrated under reduced pressure at 40° C. to half the solution volume. Make-up of the solution was performed to 10 volumes of water versus TBRE. The solution was then heated to 40-45° C. CaCl₂ (3.8 g) was added dropwise to this solution over 30-90 min at 38-45° C., forming a suspension. The suspension was then cooled to 25±5° C., stirred at 25±5° C. for 1 hr, filtered and washed with water (4×20 ml) to get a powdery compound (17.5 g dry, 93%, Chloride content 0.13% by weight).

### EXAMPLE 8

### The Effect of Number of Washings

A 1000 ml reactor equipped with a mechanical stirrer was charged with EtOH (200 mL), water (120 ml), and t-Butyl-Rosuvastatin (40 g), forming a suspension. To this suspension, NaOH 47% 1.2eq (7.6 g) was added dropwise at 25±5° C. Water (280 ml) was added, forming a mixture. The mixture was then washed with Toluene (200 ml) and stirred at 25±5° C. for half an hour.

The aqueous layer was isolated, active carbon was added, and the solution was stirred at 25±5° C. for 30 min then filtered under reduced pressure with Sinter. Thereafter the solution was concentrated under reduced pressure at 40° C. to half the solution volume. Make-up of the solution was performed to 10 volumes of water versus TBRE. The solution was heated to 40-45° C. CaCl₂ (4.1 g) was added drop wise to this solution over 1-2 hour at 38-45° C., forming a suspension. The suspension was then cooled to 25±5° C., stirred at 25±5° C. for 1 hr, filtered and washed with water (6×20 ml) to get a powdery compound.

### EXAMPLE 9

### Slurry Milled After Addition of CaCl₂

A 1000 ml reactor equipped with a mechanical stirrer was charged with EtOH (100 mL), water (60 ml), and t-Butyl-Rosuvastatin (20 g), forming a suspension. To this suspension, NaOH 47% 1.2 eq (3.8 g) was added dropwise at 25±5° C. to form a mixture. The mixture was stirred at 25±5° C. for two hours, and water (140 ml) was added. The mixture was then washed with Toluene (100 ml) and stirred at 25±5° C. for half an hour.

The aqueous layer was isolated. Thereafter the solution was concentrated under reduced pressure at 40° C. to half the solution volume. To this solution active carbon was added and the solution was stirred at 25±5° C. for 30 min. and filtered under reduced pressure with Sinter and Hyflo to eliminate the active carbon particles present in the solution. Make-up of the solution was performed to 10 volumes of water versus TBRE. The solution was heated to 40-45° C. CaCl₂ (4.13 g) was added dropwise to this solution over 30-90 min at 38-45° C., forming a suspension. The suspension was then cooled to 25±5° C. The resulting slurry was milled by wet mill (Ultra Turrax T-25 from IKA) for 10 min and stirred at 25±5° C. for 1 hr, filtered and washed with water (4±30 ml) to get a powdery compound (16.8 g dry, 90%, Chloride content 0.01% by weight)

### EXAMPLE 10

### Addition of NaBH₄

A 1000 ml reactor equipped with a mechanical stirrer was charged with EtOH (100 mL), water (60 ml), t-Butyl-Rosuvastatin (20 g), and NaBH₄ (0.1 g). To the resulting suspension, NaOH 47% 1.1 eq (3.5 g) was added dropwise at 25±5° C. to form a mixture. The mixture was stirred at 25±5° C. for two hours and then filtered under reduced pressure with a Sinter. Water (140 ml) was added and the mixture was acidified with HCl 0.1M until PH 8-10. The mixture was then washed with Toluene (100 ml) and stirred at 25±5° C. for half an hour.

The aqueous layer was isolated. To the aqueous phase active carbon was added and the solution was stirred at 25±5° C. for 30 min. The solution was filtered under reduced pressure with Sinter and Hyflo to eliminate the active carbon present. Thereafter the solution was concentrated under reduced pressure at 40° C. to half the solution volume. Make-up of the solution was performed to 10 volumes of water versus TBRE. The solution was heated to 40-45° C. CaCl₂ (4.13 g) was added dropwise to this solution over 30-90 min at 38-45° C., forming a suspension. The suspension was then cooled to 25±5° C., stirred at 25±5° C. for 1 hr, filtered and washed with water (4×20 ml) to get a powdery compound (17.3 g dry, 92%, Chloride content 0.02% by weight).

### EXAMPLE 11

### Ca(OAc)₂

A 1000 ml reactor equipped with a mechanical stirrer was charged with EtOH (100 ml), water (60 ml), and t-Butyl-Rostivastatin (20 g), forming a suspension. To this suspension, NaOH 47% (1.2 eq, 3.8 g) was added dropwise at 25±5° C. to form a mixture. The mixture was stirred at 25±5° C. for two hours and then filtered under reduced pressure with a Sinter. To this mixture water (140 ml) was added and the mixture was then washed with toluene (100 ml) and stirred at 25±5° C. for half an hour.

The aqueous layer was isolated. Thereafter the solution was concentrated under reduced pressure at 40° C. to half the solution volume. Make-up of the solution was performed to 10 volumes of water versus TBRE and the solution was heated to 40-45° C. Ca(OAc)₂ (5.9 g) was added portion-wise to this solution over 30-90 min at 25±5° C. The resulting slurry was then stirred at 25±5° C. for 1 hr, filtered and washed with water (2×20 ml) to get a powdery compound. Acetate content 0.44% and sodium content 0.17% (by ICP analysis).

### EXAMPLE 12

### Compressing of the Wet Cake

| | A level of |
|---|---|
| Description | chlorides |
| compressing the wet | 0.08 |
| cake of rosuvastatin | |
| calcium for about 1 | |
| hour in high speed | |
| centrifuge | |

A 100 liter reactor equipped with a mechanical stirrer was charged with EtOH (19.7 L), water (11.8 L), t-Butyl-Rosuvastatin (3.94 Kg), to form a suspension. To this suspension, NaOH 47% 1.1 eq (750 g) was added drop-wise at 25±5° C. to form a mixture. The mixture was stirred at 25±5° C. for two hours. Water (23.6 L) was added and the mixture was acidified by HCl 0.1M until a pH of 10.5 was obtained. The mixture was then washed with Toluene (19.7 L) and stirred at 25±5° C. for half an hour.

The aqueous layer was isolated. To the aqueous layer, active carbon (280 g) was added and resulting solution was stirred at 25±5° C. for 30 min. The solution was then filtered under reduced pressure. Thereafter the solution was concentrated under reduced pressure at 40° C. to half the solution volume. Make-up of the solution to 10 volumes of water versus TBRE was performed and the solution was then heated to 40-45° C. CaCl₂ (812 g) was added dropwise to this solution over 30-90 min at 38-45° C., forming a suspension. The suspension was then cooled to 25±5° C. and the resulting slurry was stirred at 25±5° C. for 1 hour. The solid was then filtered by-centrifuge, washed with water (4×3.94 L), and then compressed for about 1 hour in high speed (1000 rpm, 398 G). The wet cake so-obtained had a LOD of about 20%. The compound was then dried under vacuum at 30-50° C. for 36 hours to get a powdery compound (3.12 Kg dry, 87%). The level content of chloride is 0.08%.

1-12. (canceled)

13. A process of reducing formation of rosuvastatin calcium composition containing salt by-products comprising the steps of:
a) combining a catalytic amount of sodium borohydride with an aqueous reaction mixture containing a C₁ to C₄ alkyl ester of rosuvastatin;
b) adding a base to the reaction mixture to hydrolyze the ester;
c) adding a source of calcium to the hydrolyzed ester to precipitate rosuvastatin calcium.

14. The process of claim 13, wherein the composition is in the form of aggregates.

15. The process of claim 13, wherein the sodium borohydride is added to the ester before the base.

16. The process of claim 13, wherein the salt by-product is a chloride salt.

17. The process of claim 16, wherein the salt by-product is sodium chloride.

18. The process of claim 13, wherein the salt by-product is an acetate salt.

19. The process of claim 18, wherein the salt by-product is sodium acetate.

20. (canceled)

21. The process of claim 13, wherein the process results in a rosuvastatin calcium having a salt by-product content of less than about 0.1% by weight.

22. The process of claim 21, wherein the rosuvastatin calcium has a chloride content of less than about 0.1% by weight.

23. The process of claim 21, wherein the rosuvastatin calcium has an acetate content of less than about 0.1% by weight.

24. The process of claim 13, wherein the process results in a rosuvastatin calcium having a salt by-product content of less than about 0.05% by weight.

25. The process of claim 24, wherein the rosuvastatin calcium has a chloride content of less than about 0.05% by weight.

26. The process of claim 25, wherein the rosuvastatin calcium has an acetate content of less than about 0.05% by weight.

27. The process of claim 13, wherein the process results in a rosuvastatin calcium having a salt by-product content of less than about 0.03% by weight.

28. The process of claim 27, wherein the rosuvastatin calcium has a chloride content of less than about 0.03% by weight.

29. The process of claim 27, wherein the rosuvastatin calcium has an acetate content of less than about 0.03% by weight.

30. A process of reducing levels of salt by-products present in a composition of rosuvastatin calcium and the salt by products comprising the steps of:
a) providing a reaction mixture of a C₁ to C₄ alkyl ester of rosuvastatin;
b) hydrolyzing the ester with sodium or potassium hydroxide, thereby forming a rosuvastatin sodium or potassium salt;
c) adding calcium chloride or calcium acetate to obtain a composition of rosuvastatin calcium and salt by products; and
d) breaking up the composition in presence of water to reduce levels of the salt by product in the composition.

31. The process of claim 30, wherein step c) comprises adding calcium chloride or calcium acetate to the sodium or potassium salt of step b), thereby precipitating rosuvastatin calcium aggregates containing one of sodium chloride, potassium chloride, sodium acetate, or potassium acetate as a salt by-product.

32. The process of claim 31, wherein step d) comprises breaking up the rosuvastatin calcium aggregates in presence of water to reduce levels of the salt by-product in the aggregates.

33. A process of reducing formation of rosuvastatin calcium aggregates comprising the steps of:
a) providing a reaction mixture of a C₁ to C₄ alkyl ester of rosuvastatin;
b) adding a catalytic amount of sodium borohydride to the reaction mixture;
c) hydrolyzing the ester with sodium or potassium hydroxide, thereby forming a sodium or potassium salt; and
d) adding calcium chloride or calcium acetate to the sodium or potassium salt, thereby precipitating rosuvastatin calcium.

34-45. (canceled)
